(19) United States
(12) Patent Application Publication
(10) Pub. No.: US 2011/0207167 A1
(43) Pub. Date: Aug. 25, 2011

(54) METHOD OF ISOLATING A CELL FROM URINE

(76) Inventor: HARVEY L. KAUFMAN, Hudson, OH (US)

(21) Appl. No.: 12/892,131

(22) Filed: Sep. 28, 2010

Related U.S. Application Data
(60) Provisional application No. 61/338,531, filed on Feb. 19, 2010.

(51) Int. Cl. C12Q 1/04; C12N 5/09

(52) U.S. Cl. 435/34; 435/325

(57) ABSTRACT

A method of isolating a cell from a urine sample includes dialyzing the urine sample with a dialysate. The dialysate is separated from the urine sample by a dialysis membrane. The dialysis membrane excludes the passage of the cell through the dialysis membrane.

### RELATED APPLICATION

This application claims priority from U.S. Provisional Application No. 61/338,531, filed Sep. 28, 2009, the subject matter, which is incorporated herein by reference.

### TECHNICAL FIELD

This application generally relates to methods of isolating a cell from a urine sample and to methods of diagnosing disorders characterized by abnormal cell growth in a subject.

### BACKGROUND

Early detection is a key feature in treating cancer and other disorders characterized by abnormal cell growth in patients. Early detection of a disorder characterized by abnormal cell growth can allow for early therapeutic intervention, which may lead to inhibiting the progression of the disorder to an invasive and less-curable disease, and thus increase the survival rate of the subjects at risk.

Bladder cancer, for example, may be diagnosed via a cystoscopy, an invasive procedure, wherein a fiber optic device is inserted into the bladder and lesions are detected visually by a urologist. Cystoscopy is performed on patients expressing the symptom complex characteristic of bladder cancer, i.e., hematuria, pain, or urinary obstruction. Nevertheless, when symptoms appear, the tumor is usually progressed to a dangerous grade or stage.

Alternatively, in some types of cancer, such as in kidney, urethral or bladder cancers, the cancer cells can be detected in the analysis of voided urine. Cytological urinalysis can be used to detect and/or identify live and dead cells in a urine sample, including cancer cells. However, current methods of detecting cells in the urine sediment are costly and insufficient due to urine sample impurities and other sediment covering these cells which suppress the readability and accuracy rates of current analyses making it very difficult to identify individual abnormal cells. Therefore, there remains a need for a method allowing for more accurate and precise identification of cells in urine.

### SUMMARY

This application provides methods of isolating a cell from a urine sample. It was found that impurities and sediment in a urine sample from a subject can be separated from cell through the use of dialysis. Therefore, one aspect of the application provides a method of isolating a cell from a urine sample. The method includes dialyzing the urine sample with a dialysate. The dialysate is separated from the urine sample by a dialysis membrane that excludes the passage of the cell through the dialysis membrane.

In some aspects, the dialysis membrane is a molecular weight exclusion dialysis membrane. In some aspects, the molecular weight exclusion dialysis membrane can have a molecular weight cut off of about 3000 daltons to about 5000 daltons. In some aspects, the dialysate can include cell culture medium.

The method can further include the step of maintaining the isolated cell in culture. The method can also include the steps of staining the cell with a cytological staining agent and identifying the cell using microscopic examination. In some aspects, the cytological staining agent can include a morphological stain. The morphological stain can be selected from the group consisting of May-Griinwald-Giemsa stain, Giemsa stain, Papanicolau stain, Hematoxylin-Eosin stain and DAPI stain.

In other aspects, the cell can include an abnormal cell. The abnormal cell can include a neoplastic cell, a tumor cell, a circulating tumor cell, a pre-cancerous cell, a cancerous cell, and a cancer cell. The cancer cell can include, for example, a kidney cancer cell, a bladder cancer cell, or an urethal cancer cell. The cancer cell can also include a carcinoma cell. A carcinoma cell can include a transitional cell carcinoma, a squamous cell carcinoma, an adenocarcinoma, a small cell carcinoma or associated metastases.

In another aspect, a diagnostic assay for detecting a disorder characterized by abnormal growth in a subject is provided. The diagnostic assay includes obtaining a urine sample from the subject, dialyzing the urine sample with a dialysate. The dialysate separated from the urine sample by a dialysis membrane that excludes the passage of a cell through the dialysis membrane. The dialyzed sample is examined and the presence of an abnormal cells in the dialyzed sample is correlated with a disorder characterized by abnormal cell growth.

In some aspects, the dialysis membrane is a molecular weight exclusion dialysis membrane. In some aspects, the molecular weight exclusion dialysis membrane can have a molecular weight cut off of about 3000 daltons to about 5000 daltons. In some aspects the dialysate can include cell culture medium.

In some aspects, the step of examining the dialyzed sample includes staining the dialyzed sample with a cytological staining agent and detecting the presence of an abnormal cell using microscopic examination. In some aspects, the cytological staining agent can include a morphological stain, the morphological stain selected from the group consisting of May-Griinwald-Giemsa stain, Giemsa stain, Papanicolau stain, Hematoxylin-Eosin stain and DAPI stain.

In other aspects, an abnormal cell can include a neoplastic cell, a tumor cell, a circulating tumor cell, a pre-cancerous cell, a cancerous cell, and a cancer cell. In some aspects, the presence of a cancer cell in the dialyzed sample is indicative of a positive diagnosis of cancer. The cancer cell can include, for example, a kidney cancer cell, a bladder cancer cell, or an urethal cancer cell. The cancer cell can also include a carcinoma cell. A carcinoma cell can include a transitional cell carcinoma, a squamous cell carcinoma, an adenocarcinoma, a small cell carcinoma or associated metastases. In some aspects, the presence of a carcinoma cell in the dialyzed sample is indicative of a positive diagnosis of carcinoma.

### DESCRIPTION OF DRAWINGS

The foregoing and other features of the application will become apparent to those skilled in the art to which the application relates upon reading the following description with reference to the accompanying drawings, in which:

FIG. 1 is a photomicrograph ×1000 under oil immersion of: A) Normal human epithelial cells found in the urine, originating in the kidney, bladder or urethra; and B) Normal human epithelial cells found in the urine, originating in the kidney, bladder or urethra with a heavier biological stain than A.

FIG. 2 is a photomicrograph ×1000 under oil immersion of: A) Cancerous tumor tissue, found in the urine, originating in the kidney, bladder or urethra; and B) Cancerous epithelial cell separated from tissue of A.

FIG. 3 is a photomicrograph ×1000 under oil immersion of: A) Field of normal epithelial cells mixed with cancerous epithelial cells. Note cancer cells apparent in center of photo; note one de-nucleated epithelial cell apparent in upper right; and B) One cancerous epithelial cell (upper center); one normal epithelial cell (lower center). Note field of view clarity.

FIG. 4 is a photomicrograph ×1000 under oil immersion of a cluster of epithelial cancer cells approaching Stage 4; Note two severely deformed nuclei and severely deformed cell shape (cell origin: kidney, bladder or urethra).

FIG. 5 is a flow chart illustrating an exemplary method of isolated a cell from a urine sample.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the application.

The term “subject”, “patient” and “individual” are used herein interchangeably. They refer to a human or another mammal (e.g., primate, dog, cat, goat, horse, pig, mouse, rat, rabbit, and the like), that can be afflicted with a disorder characterized by abnormal cell growth, such as bladder cancer but may or may not have the disease. In many embodiments, the subject is a human being.

As used herein, the term “detecting the presence or absence of an abnormal cell” refers to the detection of abnormal cells (e.g., neoplastic or cancerous cells) in a urine sample. The detecting may be carried out using any suitable examination method, including, but not limited to, those disclosed herein.

As used herein, the term “diagnosis” refers to a process aimed at determining if an individual is afflicted with or has an increased risk of a disorder, disease or ailment. In the context of the application, “diagnosis of a disorder characterized by abnormal cell growth” refers to a process aimed at: determining if a subject is likely to or has an increased risk to develop a disorder characterized by abnormal cell growth, determining if a subject is afflicted with or has increased risk of a disorder characterized by abnormal cell growth and/or even the impact of the presence of a disorder characterized by abnormal cell growth (e.g., as determined by the diagnostic methods of the application) on a subject's future health (e.g., expected morbidity or mortality).

The term “urine sample” is used herein in its broadest sense. A urine sample may be obtained from a subject (e.g., a human) or from components (e.g., tissues) of a subject. Frequently, the sample will be a “clinical sample”, i.e., a sample derived from a patient.

As used herein, the term “indicative of a positive diagnosis”, when applied to an isolated abnormal cell, refers to the identification of an abnormal cell which is diagnostic of a disease or disorder characterized by abnormal cell proliferation such that the identification of a particular abnormal cell is found significantly more often in subjects with a disease or disorder characterized by abnormal cell proliferation than in patients without the disease or disorder (as determined using routine statistical methods setting confidence levels at a minimum of 95%).

The term “tumor”, as used herein, refers to all neoplastic cell growth and proliferation, whether malignant or benign, and all pre-cancerous and cancerous cells and tissues.

The term “chemotherapeutic agent” can refer to any chemical compound useful in the treatment of a neoplastic disease, such as cancer.

The term “membrane” may further refer to an interface. Unless specified otherwise, membranes may take the form a solid, liquid, or gel, and may or may not have a distinct lattice, non cross-linked structure, or cross-linked structure.

As used herein, the term “semi-permeable membrane” means a membrane that is substantially selective based on size or molecular weight. Thus, a semi-permeable membrane substantially passes sedimentary particles as well as other dissolved ions, inorganic and organic compounds of a first molecular weight or size found in a urine sample, while substantially blocking passage of cells or cell particles of a second molecular weight or size, greater than the first molecular weight or size.

This application generally relates to methods for isolating cells from a urine sample and for diagnosing disorders in a subject characterized by abnormal cell proliferation, including but not limited to cancer. It was found that numerous cell types, including live and dead cancer cells, can be isolated or removed from the impurities found in a urine sample using a filtration and dialysis process. In general, the method described herein includes the use of dialysis to separate or isolate one or more cells from a urine sample. The methods include separating salts, color, crystals, enzymes and other urine sediment from a urine sample in order to more accurately isolate, detect and identify abnormal cells in a urine sample. The application also relates to a method of culturing abnormal cells and to determining the effectiveness of a chemotherapeutic treatment in a subject having a disorder characterized by abnormal cell proliferation.

Therefore, one aspect of the application relates to a method of isolating a cell from a urine sample for subsequent use in research, diagnostic testing and/or treatment. The method includes dialyzing the urine sample with a dialysate separated from the urine sample by a dialysis membrane.

In some embodiments, a urine sample can be obtained from a subject. Any method or combination of methods for obtaining a urine sample from a subject may be employed. For example, a urine sample can be obtained through the use of a urine collection device (UCD). A UCD used herein can include any utensil that allows an individual to empty his or her bladder into a container hygienically and without spilling urine. In addition, special UCDs exist for the collection of urine samples for subsequent urinalysis. They range from a simple plastic cup to elaborate devices designed to collect specific volumes or types of urine samples at various points in the micturition process.

A related type of device can be used for urine collection in bedridden and unconscious patients. This type of UCD usually includes a catheter inserted directly into the urethra of the patient in order to collect all urine as it is produced or whenever micturition occurs.

In some embodiments, a urine sample can be a midstream urine sample obtained from a subject to avoid possible contamination of the forestream urine. In some examples, there is enough urine sample to accurately and reliably detect the presence or absence of abnormal cells in the urine of the subject as described below. However, multiple urine samples may be taken from the subject in order to obtain a representative sampling or urine from the subject.

In an exemplary embodiment, about 10 to about 30 ml of a midstream urine sample is collected from a subject. In some embodiments, the inventive methods are performed on the urine sample itself without or with limited processing of the sample. In some embodiments, a preservative may be added to a collected sample. For example, 2-isopropyl-5-methylphenol, also known as Thymol, can be added to the collected sample. In other embodiments, a 24 hour collection can be obtained from a subject.

In certain embodiments, the urine sample may be refrigerated after being obtained from a subject. For example, a collected sample can be stored at +2° C. to +10° C. for about 2 hours post collection. The collected sample can be stored in any laboratory refrigerator that complies with regulations from agencies, such as the U.S. Food and Drug Administration (FDA) that are designed to provide specific levels of temperature control and a uniform temperature throughout the chamber.

A urine sample as described herein can contain a relatively small number of cells or can contain a large number of cells. Typically, a urine sample containing one or more cells will also contain unwanted salts, color, crystals, enzymes and other urine sediment. For example, components of a urine sample can include, but are not limited to urea, uric acid, hippuric acid, amino acids, calcium, magnesium, chloride, sodium, potassium, creatine, creatinine, ammonia, phosphates, sulfates, urobilinogen, casts and other dissolved ions, inorganic and organic compounds.

Therefore, in order to isolate a cell from the unwanted components of urine, the urine sample is dialyzed. Any method of dialysis or any dialysis apparatus known to the skilled artisan may be employed in the method described herein. In certain embodiments, the step of dialyzing the urine sample employs a semi-permeable size-exclusion membrane that retains cells but allows urine sediment and small molecules to diffuse freely through the membrane. For example, a semi permeable membrane can include a molecular weight exclusion dialysis membrane having pores of a known size range that are large enough to let urine sediment and small molecular weight urine components pass through, but that are small enough to exclude the passage of a cell through the membrane. In an exemplary embodiment, a molecular weight exclusion dialysis membrane can have a molecular weight cut off of about 3000 to about 5000 daltons.

In some embodiments, molecular weight exclusion dialysis tubing or a cassette having a molecular weight exclusion dialysis membrane can be employed to dialyze the urine sample. In some embodiments, all or a portion of the urine sample obtained from a subject can be loaded into a dialysis cassette, such as, but not limited to a Slide-a-Lyzer dialysis cassette (Pierce, Rockford, Ill.), which is described in U.S. Pat. Nos. 5,503,741 and 6,039,871, and incorporated herein by reference.

Once loaded with a urine sample, the dialysis apparatus can be placed in contact with a dialysate (e.g., a growth medium or culture medium). In general, any dialysate allowing for the dialysis of a urine sample that results in the isolation of one or more cells from the urine sample can be employed herein. In some embodiments, it may be advantageous to have a small urine sample volume relative to a large dialysate volume, in order to maximize the concentration differential. For example, the dialysate, can be about 10 to about 300 times the volume of the sample. This creates and maintains a concentration differential across the membrane.

Once the liquid-to-liquid interface (urine sample on one side of the membrane and dialysate on the other) is initiated, all molecules will then try to diffuse in either direction across the membrane in order to reach equilibrium. Dialysis (diffusion) will stop when equilibrium is achieved. Because each sample is different, the optimum time of dialysis may be derived empirically. Generally the rate of dialysis slows as equilibrium approaches, therefore in some embodiments, the dialysate can be changed after several hours to re-create the concentration differential that drives the dialysis process. In some embodiments, dialysis may continue overnight to yield a more complete dialysis.

In some embodiments, the dialysate can include any cell culture medium designed to support the growth of mammalian cells. The urine sample may be dialyzed for the amount of time required to significantly remove impurities and urine sediment from the urine sample allowing the remaining cells to be clearly visible using microscopic analysis.

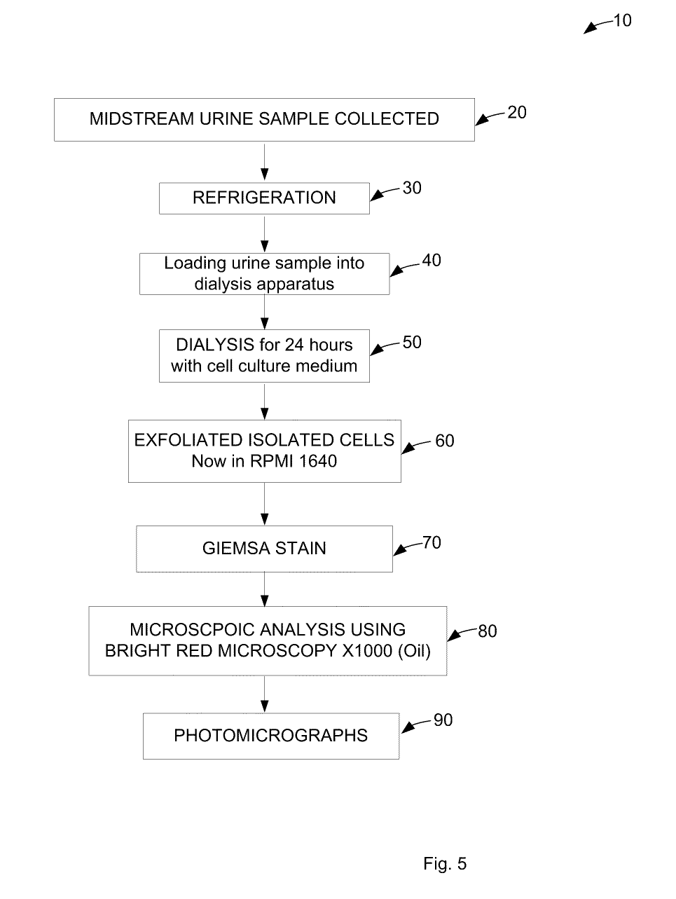

FIG. 5 is a flow diagram illustrating an example of a method 10 of isolating a cell from a urine sample. In the example, at 20, a midstream urine sample is collected. The urine sample is then refrigerated at 30. Following refrigeration, the urine sample is loaded into a dialysis apparatus at 40, such as a size exclusion dialysis cassette. After the urine sample is loaded in the dialysis apparatus, dialysis is performed for 24 hours with cell culture medium at 50, resulting in isolated exfoliated cells in the cell culture medium at 60. The isolated cells are then stained with Giemsa stain at 70, microscopically analyzed using bright red microscopy ×1000 (Oil) at 80, and photomicrographs are produced at 90.

It is further contemplated that the urine sample having been cleaned and the residual urine replaced with standard cell culture media during dialysis allows the isolated cells to be grown and maintained in the culture media. Thus, in some embodiments, one or more isolated cells are removed from the dialysis apparatus and placed in an incubator at 37° C. with humidity to culture the isolated cells.

As illustrated in the Examples below, it is also contemplated that following dialysis, clear and well established cell types are concentrated and clearly identifiable through the use of cell staining. It is further contemplated that the isolation, examination and identification of abnormal cells in a urine sample from a subject can be correlated with the subject having a disorder characterized by abnormal cell growth. Therefore, another aspect of the application provides a diagnostic assay for detecting a disorder in a subject characterized by abnormal cell growth.

Abnormal cells can be detected and identified in urine samples by staining the specimens with a variety of cellular staining agents and associated methods. Staining methods for use herein include cytological stains, which are based on the morphology of the cells, along with immunohistochemistry and activity stains, which rely on the presence or absence of antigens and enzymatic activities in the abnormal cells, and DNA and chromosome stains, which detect the presence of chromosomal abnormalities often associated with a proliferative disease, such as cancer. In some embodiments, the staining methods are designed to differentiate abnormal neoplastic cells, cancerous cells, or pre-cancerous cells from any normal cells shed from the subject and collected as part of the urine sample obtained from the subject.

In some embodiments, the one or more isolated cells can be examined in accordance with well known cell staining procedures by sequentially and/or simultaneously exposing the stained cells to at least one imaging modality to thereby identify the abnormal cells in the urine sample. It will be appreciated that a cell is considered as an abnormal cell if it exhibits abnormal findings according to a staining method.

For example, urine samples can be centrifuged in the presence of a morphology preserver, to prepare cytospin slides for at least one type of stain. Slides are then subjected to a stain, such as for example a cytological stain (e.g., May-Griinwald Giemsa, Giemsa, Papanicolau or Hematoxylin-Eosin), which labels the nuclear and cytoplasmic compartments of the cell and enables the screening of morphological abnormalities typical to neoplastic and cancerous cells. The stained cells can then be scanned using an imaging apparatus using an imaging modality suitable for the stain. For example, if May-Griinwald-Giemsa stain is employed then a bright field modality is used.

In some aspects, isolated cells with abnormal morphology are identified and their images are then captured and saved. In some embodiments a photomicrograph is produced using the well known methods to further facilitate accurate evaluation by technicians and other medical practitioners.

Following is a non-limiting description of a number of staining procedures and approaches for visualizing such stains, which can be utilized in a diagnostic assay for detecting a disorder characterized by abnormal growth in a subject.

### Morphological Stains

Morphological stains bind non-specifically to cell compartments rendering them visible for microscopic observation. Examples include but are not limited to May-Griinwald-Giemsa stain, Giemsa stain, Papanicolau stain, Hematoxylin-Eosin stain, DAPI stain and the like.

Morphological staining can be effected by simple mixing, diluting and washing laboratory techniques and equipment. Following the application of the appropriate stain, the microscopic slides containing stained cells can be viewed under a microscope equipped with either a bright or a dark field source of light with the appropriate filters according to manufacturer's instructions. For example, May-Griinwald-Giemsa stain, Giemsa stain, Papanicolau stain and/or Hematoxylin-Eosin stain can be viewed using bright field modality. On the other hand, DAPI stain is viewed using a dark field modality with a UV lamp.

### Immunological Stains

Immunological staining is based on the binding of labeled antibodies to antigens present on the cells. Examples of immunological staining procedures include but are not limited to, fluorescently labeled immunohistochemistry, radiolabeled immunohistochemistry and immunocytochemistry. Immunological staining is preferably followed by counterstaining the cells with a dye which binds to nonstained cell compartments. For example, if the labeled antibodies bind to antigens present on the cell cytoplasm, a nuclear stain (e.g., Hematoxylin-Eosin stain) is an appropriate counterstaining. Antibody labeling can be effected using numerous labeling modes known in the art.

For example, antibodies can be conjugated to a fluorescent dye (e.g., fluorescent immunohistochemistry) in which case visualization is direct using a fluorescent microscope and a dark field image modality. Antibodies can also be radiolabeled with certain isotopes, in which case bound antibodies are retrieved following the development of a photographic emulsion which results in localized silver grains in cells containing bound antibodies. These silver grains can be further viewed under a microscope using a bright field modality.

Alternatively, antibodies can be conjugated to an enzyme (e.g., horseradish peroxidase (HRP)) in which case, upon binding to a chromogenic substrate specific to the conjugated enzyme, the enzyme catalyzes a reaction in which the chromogenic substrate becomes detectable when viewed under a light or a fluorescent microscope.

### Activity Stains

According to this method, a chromogenic substrate is applied on the cells containing an active enzyme. The enzyme catalyzes a reaction in which the substrate is decomposed to produce a chromogenic product visible by a light (e.g., bright field modality) or a fluorescent microscope (e.g., dark field modality). Examples of commonly practiced activity staining procedures include but are not limited to cytochemical stain and substrate binding assays.

Substrate binding assays utilize endogenous substrates in order to activate a chromogenic dye bound to an ectopically introduced enzyme. In this method, once the enzyme binds to its natural substrate on the cell, a conformational change within the enzyme molecule activates the conjugated dye in such a way that a chromogenic product will deposit on the cell. The chromogenic product can be further viewed under a light microscope using bright field modality or under a fluorescent microscope using dark field modality.

### Cytogenetical Stains

Cytogenetical stains are useful for karyotyping and identifying major chromosomal aberrations. Conventional banding techniques include G-banding (Giemsa stain), Q-banding (Quinacrine mustard stain), R-banding (reverse Giemsa), and C-banding (centromere banding). Chromosomes are typically examined by bright-field microscopy after Giemsa staining (G-banding), or by fluorescence microscopy using dark field modality after fluorescence staining (R-banding), to reveal characteristic light and dark bands along their length. Careful comparison of a subject's banding pattern with those of normal chromosomes can reveal abnormalities such as translocations (exchange of genetic material between or within chromosomes), deletions (missing chromosome(s) or fragment(s) thereof), additions, inversions and other defects that cause deformities and genetic diseases

### In Situ Hybridization Stains

In situ hybridization is a useful method of detecting major and/or minor chromosomal aberrations. In this method labeled nucleic acid probes are denatured and applied on fixed and denatured cells in either the metaphase or the interphase stages of cell cycle. The attachment of the labeled probes to their genomic counterparts reveals specific signals, which can be detected using a microscope. Examples for in situ hybridization include, but are not limited to fluorescent in situ hybridization (FISH), radiolabeled in situ hybridization, Digoxigenin labeled in situ hybridization and biotinylated in situ hybridization.

Numerous nucleic acid labeling techniques are known in the art. For example, a fluorescent dye can be covalently attached to either the 5′ or 3′ end of a nucleic acid probe. Following hybridization, the labeled probe can be directly retrieved using a fluorescent microscope and a dark field modality.

Alternatively, a nucleic acid probe can be directly labeled with a radioactively labeled nucleotide, such as [35S]ATP. In this case the labeled nucleotide can be incorporated to the nucleic acid probe by conventional labeling techniques known to those skilled in the art of molecular biology. Labeling techniques include, but are not limited by, Nick Translation, Random Primed Labeling, End Labeling with a polynucleotide kinase etc. Following hybridization, the labeled nucleic acid probes are retrieved by the development of a photographic emulsion which produces dark silver grains that can be further viewed under a light microscope using bright field modality.

Optionally, a nucleic acid probe can be prepared by incorporating a Digoxigenin (DIG) labeled nucleotide to the nucleic acid probe. Digoxigenin labeled nucleotides are prepared according to the labeling techniques described herein. Following hybridization, an anti DIG antibody is applied on the cells. Anti-DIG antibodies can be directly labeled with a fluorescent dye in which case the hybridization signal is viewed under a fluorescent microscope using dark field modality or they can be conjugated to an enzyme (e.g., HRP), in which case upon the addition of a chromogenic substrate will produce a color that can be further viewed under a microscope using bright field or dark field modalities.

The nucleic acid probes can be also conjugated to a biotin molecule at the 5′ or 3′ end of the nucleic acid probe. In this case, following hybridization, an avidin or a streptavidin molecule is further applied on the cells. The avidin or streptavidin molecules used herein can be directly labeled with a fluorescent dye or can be conjugated to an enzyme which will further produce a chromogenic product once the appropriate substrate is employed. It will be appreciated that fluorescent avidin or streptavidin molecules are further detected under a fluorescence microscope using a dark field modality. However, if a chromogenic product is to be produced the in situ hybridization stained slides are usually viewed under a light microscope using a bright field modality.

### DNA Stains

DNA stains are based on the attachment of fluorescent dyes to DNA molecules in order, for example, to quantitate the amount of DNA present in the cells at a specific time. For example, during replication, the amount of DNA chromosome per cell is multiplied, i.e., from 2N to 4N chromosomes.

Examples for DNA stains include, but are not limited to 4′,6-diamidino-2-phenylindole (DAPI), Propidium Iodide (PI) and Ethidium bromide which can be viewed under a fluorescence microscope using a dark field modality.

Other methods of detecting abnormal cells utilize the presence of chromosomal aberrations in the cells. In particular, the deletion or multiplication of copies of whole chromosomes or chromosomal segments, and higher levels of amplifications of specific regions of the genome are common occurrences in cancer.

Chromosomal aberrations are often detected using cytogenetic methods such as Giemsa-stained chromosomes (G-banding) or fluorescent in situ hybridization (FISH). FISH is considered an advanced approach over cytogenetic and is often used for the detection of bladder cancer.

In some embodiments, the imaging modality allows for microscopic examination of the stained cells. Any method of microscopic examination known to the skilled artisan may be employed. A stained cell isolated from a urine sample can be examined under a phase-contrast microscope using a hemocytometer or similar device known in the relevant art. For example a Neubauer counting chamber can be used where the stained urine sample is under the cover slide, in the upper segment formed by the H-shaped grooves.

In some embodiments, bright field or inverted microscopy can be used to examine the stained urine sample. In other embodiments, the stained cells of the urine sample are photographed and counted while being microscopically examined.

Typically, urine samples, stained by any of the methods described herein, are manually read and interpreted by a lab technician or a pathologist, in order to determine the presence or absence of an abnormal cell in the urine sample and further identify one or more types of abnormal cells present in the urine sample.

Abnormal cells, which can be detected by the method described herein, include cells that divide at an abnormal (i.e., increased) rate, such as neoplastic cells, tumor cells, pre-cancerous cells, and cancerous or cancer cells. In some embodiments, the abnormal cells detected in a urine sample in accordance with the method are cancerous cells. Cancerous or cancer cells are cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within the body, or may be a non-tumorigenic cancer cell, such as a leukemia cell. Cancerous cells can be associated with many kinds of cancers including, but not limited to carcinoma, leukemia, lymphoma, brain cancer, cerebrospinal cancer, bladder cancer, prostate cancer, breast cancer, cervix cancer, uterus cancer, ovarian cancer, kidney cancer, esophagus cancer, lung cancer, colon cancer, melanoma, neuroblastoma, and pancreatic cancer. More particular examples of such cancers specific to bladder and/or urinary tract carcinoma include: urothelial or transitional cell carcinoma; squamous cell carcinoma, adenocarcinoma, small cell carcinoma and associated metastases. Urothelial carcinoma are further classified as: invasive or noninvasive and papillary or flat. Papillary urothelial tumors are further classified as benign, low malignant potential and high malignant potential.

In some embodiments a scoring methodology can be used to identify a specific cell type in the urine sample. For example, a stained cytology slides can be scored according to the following categories: Class I-normal, Class II-inflammation, Class III-suspicious for malignancy, and Class IV-malignant. In an exemplary embodiment, a cluster of epithelial cells approaching Stage 4 can be identified. For a review of cancer scoring methodologies see American Joint Committee on Cancer: AJCC Cancer Staging Manual, 7th ed. Edge, S. B.; Byrd, D. R.; Compton, C. C.; Fritz, A. G.; Greene, F. L.; Trotti, A. (Eds.): Springer; (Oct. 6, 2009), which is incorporated herein by reference.

As is illustrated in the Examples it was found that the method described herein increases the ability to accurately detect abnormal cells (e.g., cancerous tissue and cells) in a urine sample obtained from a subject. Thus, the methods described herein increase the information, which can be obtained from a urine sample and therefore improve the accuracy of detection of abnormal cells in the sample. Therefore, the detection of abnormal cells in urine samples is an important tool for diagnosing a disorder characterized by abnormal cell growth in a subject.

In some embodiments, the numbers and types of cells detected using the method can provide information to a skilled practitioner correlating to a specific diagnosis. For example, the presence of a carcinoma cell in a urine sample can be indicative of a positive diagnosis of carcinoma in the subject. Thus, another aspect of the application relates to a diagnostic assay for detecting a disorder characterized by abnormal cell growth in a subject. As used herein, the phrase “detecting a disorder characterized by abnormal cell growth in a subject” refers to detecting the presence of abnormal cells in cells derived from the subject, i.e., in urine samples obtained from the subject. The method is effected by obtaining a urine sample from the subject and processing the sample as described above in order to detect the presence or absence of abnormal cells in the sample.

The method can be utilized for diagnosing numerous types of disorders characterized by abnormal cell growth. For example, it will be appreciated that positive identification of a carcinoma cell in a urine sample can correlate with the subject having carcinoma. Therefore, the method of detecting a carcinoma cell in a urine sample can be used for diagnosing individuals having early to late stages of cancer (e.g., bladder cancer).

In certain embodiments, the disorder characterized by abnormal cell growth is a carcinoma. The term “carcinoma” refers to a malignant epithelial neoplasm which invades the surrounding tissue and metastasizes to distant regions of the body. Non limiting examples of carcinomas that can be diagnosed include transitional cell carcinomas (TCC), squamous cell carcinomas (SCC), adenocarcinoma, carcinosarcoma.

In some embodiments, the method can be used for diagnosing TCC of the bladder since even the low grade tumors include transitional epithelial cells with atypical morphology, which can be detected in a urine sample using an isolation/concentration approach described herein. However, it will be appreciated that the detection of TCC in a urine sample can also suggest the presence of carcinoma in situ, TCC of the kidney and/or TCC of the ureter.

In other embodiments, the method further allows the determination of the point of origin in the subject's body of a detected abnormal cell. As shown in the Example below, a circulating tumor cell (e.g., a breast cancer tumor cell) can be identified and correlated to the site of tumorigenesis in the subject (e.g., the subject's breast).

It is further contemplated that a diagnostic method described herein can be employed by a practitioner sequentially and/or simultaneously in addition to standard urinalysis of the subject's urine sample in order to diagnose a disorder characterized by abnormal growth in a subject. Non-limiting examples of a typical medical urinalysis includes a description of urine color and measurements of specific gravity, pH, ketone bodies, blood urea nitrogen, proteins, nitrites, urobilinogen, bilirubin, RBC destruction (Icotest), glucose, hemoglobin, RBC number, WBC number, hCG. For a review of urinalysis, see Simerville J A, Maxted W C, Pahira J J (March 2005). “Urinalysis: a comprehensive review”. American family physician 71(6): 1153-62, which is incorporated herein by reference.

Based upon the detection of one or more abnormal cells in a urine sample obtained from a subject and the related diagnosis, a corresponding therapy regimen can be tailored to the needs of the subject by a skilled practitioner. Where a subject is identified as falling into a subgroup whose diagnoses includes squamous cell cancer, for example, an appropriate therapy can then be administered to the subject. Examples of therapies that a subject diagnosed with a disorder characterized by abnormal growth may receive include any one or combination of surgery, radiation therapy, photodynamic therapy, or chemotherapy.

As discussed above, cells isolated from a urine sample obtained from a subject can be cultured in a cell culture medium following dialysis. Thus, in another embodiment, one or more abnormal cells in a urine sample obtained from a subject are employed in a method of determining the effectiveness of a therapeutic agent for the treatment of a disease characterized by abnormal cell growth in the subject. The term “determining the effectiveness” refers to determining the ability of an agent or combination of agents to inhibit abnormal cell activity, such as tumor inhibitory activity. Also as used herein, a treatment regiment is determined to be effective if it achieves a selected range of probability of response of clinical success. The meaning of response and clinical success will vary according to the clinical setting. For example, it can range from eradication of clinically apparent cancer, to stabilization of or reduction in tumor load, or to providing enhance terminal quality of life if other measures have failed.

The application also provides novel methods for selecting chemotherapeutic agents, or combinations of agents (simply “agents” or “combinations”), for a particular disorder, such as cancer, characterized by abnormal growth afflicting a particular patient. The agents or combinations comprise a plurality, which may be a plurality of different agents suspected to be effective against the same or a different disorder, or a plurality of agents suspected to be effective against the same disorder, but heretofore, not appreciated as providing a synergistic effect. A preferred plurality comprises 2 agents, 3 agents or 4 agents, which may be tested individually or as combinations. More preferred are 4 agents, 5 agents or 6 agents, again individually or in combinations. However, as is appreciated by those skilled in the art, the methods and systems described herein allow for the testing of even higher numbers of agents or combinations of agents with relative ease, as compared to conventional testing modalities.

As used in the present specification, the “agents” which are evaluated for cell growth and tumor inhibitory activity in the in vitro chemo-sensitivity/resistance assays as potential cancer therapeutics are intended to encompass any agents, which either alone or in combination with another substance, demonstrate cell growth or tumor inhibitory activity. Accordingly, the “agents” include any substance which, whether alone or in combination with another substance is cytostatic or cytotoxic to a neoplastic cell, cancerous cell, pre-cancerous cell, cancer cell, and/or tumor cell. Hence, the agents include but are not limited to known anti-cancer drugs, untested potential anticancer drugs, compounds having anti-angiogenic, anti-oncogene, anti-growth factor or receptor or membrane perturbing activity as well as other compounds such as aptamers, siRNAs, cytokines, hormones, enzymes, and the like, which when administered to a subject with another agent are cell cytostatic or cytotoxic although they may not have such activity when administered alone.

In some embodiments, the selection methods use data from assays of the sensitivity/resistance (simply “assays”) of the cells both of the particular patient's cancer and also of the cells of prior similar cancers which afflicted previous similar patients. The former data is referred to as “actual” data, while the latter data is referred to a “reference” data. The application further provides computer systems that include methods and programs implementing the selection methods.

Any in vitro assay method that has demonstrated correlation with clinical results can be employed. The assays are conducted in the laboratory on samples of cells of a particular type obtained from a particular patient. In one embodiment, the preferred assay method is the ATP-TCA assay. However, any assay method can be used that demonstrates correlation with clinical experience comparable to the ATP-TCA assay by, for example, demonstrating directly good correlation with the preferred ATP-TCA assay itself. Assay data principally includes several observations of the degree of tumor inhibition at several drug concentrations. Drug concentration can be recorded as a percent of the test drug concentration (TDC) for the particular drug as conventionally understood and determined by one of skill in the art. Tumor inhibition can be expressed as a percent of inhibition. Alternatively, cell growth and tumor inhibition can be expressed as a cell viability percentage, or in the case of the preferred ATP-TCA assay, percent of ATP concentration with respect to untreated cells. These two manners of expression are completely equivalent since percent cell growth or tumor inhibition is in fact determined as one minus percent cell viability.

In some embodiments, the agent can be a chemotherapeutic agent. Any chemotherapeutic agent, or combination of agents can be contacted with a subject's abnormal cell type in culture in order to measure the agent(s) effectiveness against the particular cell type. Non-limiting examples of chemotherapeutic agents employed can include alkylating agents, such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin; dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE, FILDESIN); dacarbazine; mannomustine; mitobronitol; mitolactol; pipobroman; gacytosine; arabinoside (“Ara-C”); thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, Ill.), and TAXOTERE doxetaxel (Rhone-Poulenc Rorer, Antony, France); chloranbucil; gemcitabine (GEMZAR); 6-thioguanine; mercaptopurine; methotrexate; platinum analogs such as cisplatin and carboplatin; vinblastine (VELBAN); platinum; etoposide (VP-16); ifosfamide; mitoxantrone; vincristine (ONCOVIN); oxaliplatin; leucovovin; vinorelbine (NAVELBINE); novantrone; edatrexate; daunomycin; aminopterin; ibandronate; topoisomerase inhibitor RFS 2000; difluoromethylornithine (DMFO); retinoids such as retinoic acid; capecitabine (XELODA); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX an abbreviation for a treatment regimen with oxaliplatin (ELOXATIN) combined with 5-FU and leucovovin.

Also included in the definition of chemotherapeutic agents are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX tamoxifen), EVISTA raloxifene, droloxifene, 4-hydroxytamoxifen, trioxifene, keoxifene, LY117018, onapristone, and FARESTON toremifene; anti-progesterones; estrogen receptor down-regulators (ERDs); agents that function to suppress or shut down the ovaries, for example, leutinizing hormone-releasing hormone (LHRH) agonists such as LUPRON and ELIGARD leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE megestrol acetate, AROMASIN exemestane, formestanie, fadrozole, RIVISOR vorozole, FEMARA letrozole, and ARIMIDEX anastrozole. In addition, such definition of chemotherapeutic agents includes bisphosphonates such as clodronate (for example, BONEFOS or OSTAC), DIDROCAL etidronate, NE-58095, ZOMETA zoledronic acid/zoledronate, FOSAMAX alendronate, AREDIA pamidronate, SKELID tiludronate, or ACTONEL risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE vaccine and gene therapy vaccines, for example, ALLOVECTIN vaccine, LEUVECTIN vaccine, and VAXID vaccine; LURTOTECAN topoisomerase 1 inhibitor; ABARELIX rmRH; lapatinib ditosylate (an ErbB-2 and EGFR dual tyrosine kinase small-molecule inhibitor also known as GW572016); and pharmaceutically acceptable salts, acids or derivatives of any of the above.

In some embodiments, the chemotherapeutic agent can include a growth inhibitory agent. A “growth inhibitory agent” when used herein refers to a compound or composition which inhibits growth of a cell, especially a GOUTC-expressing cancer cell, either in vitro or in vivo. Thus, the growth inhibitory agent may be one which significantly reduces the percentage of GOUTC-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE, Rhone-Poulenc Rorer), derived from the European yew, is a semisynthetic analogue of paclitaxel (TAXOL, Bristol-Myers Squibb). Paclitaxel and docetaxel promote the assembly of microtubules from tubulin dimers and stabilize microtubules by preventing depolymerization, which results in the inhibition of mitosis in cells.

In another aspect, the application provides kits comprising materials useful for carrying out diagnostic methods described herein. The diagnosis and cell isolating and identification procedures described herein may be performed by diagnostic laboratories, experimental laboratories, or practitioners.

Materials and reagents for characterizing urine samples, diagnosing disorders characterized by abnormal cell growth, and/or detecting and identifying abnormal cells obtained from a subject according to the methods may be assembled together in a kit. In certain embodiments, a kit includes at least one reagent or apparatus used to obtain, dialyze, store, stain, culture or assay a urine sample, and instructions for using the kit according to the method.

The reagents may be supplied in a solid (e.g., lyophilized) or liquid form. The kits may optionally comprise different containers (e.g., vial, ampoule, test tube, flask or bottle) for each individual buffer and/or reagent. Each component will generally be aliquoted in its respective container or provided in a concentrated form. Other containers for conducting certain steps of the disclosed methods may also be provided. The individual containers of the kit can be maintained in close confinement for commercial sale.

Instructions for using the kit, according to one or more methods described herein, may comprise instructions for assaying the urine sample obtained from the subject, and/or for performing the test, instructions for interpreting the results. As well as a notice in the form prescribed by a governmental agency (e.g., FDA) regulating the manufacture, use or sale of pharmaceuticals or biological products.

The following examples are for the purpose of illustration only and are not intended to limit the scope of the claims, which are appended hereto.

### EXAMPLES

### Example 1

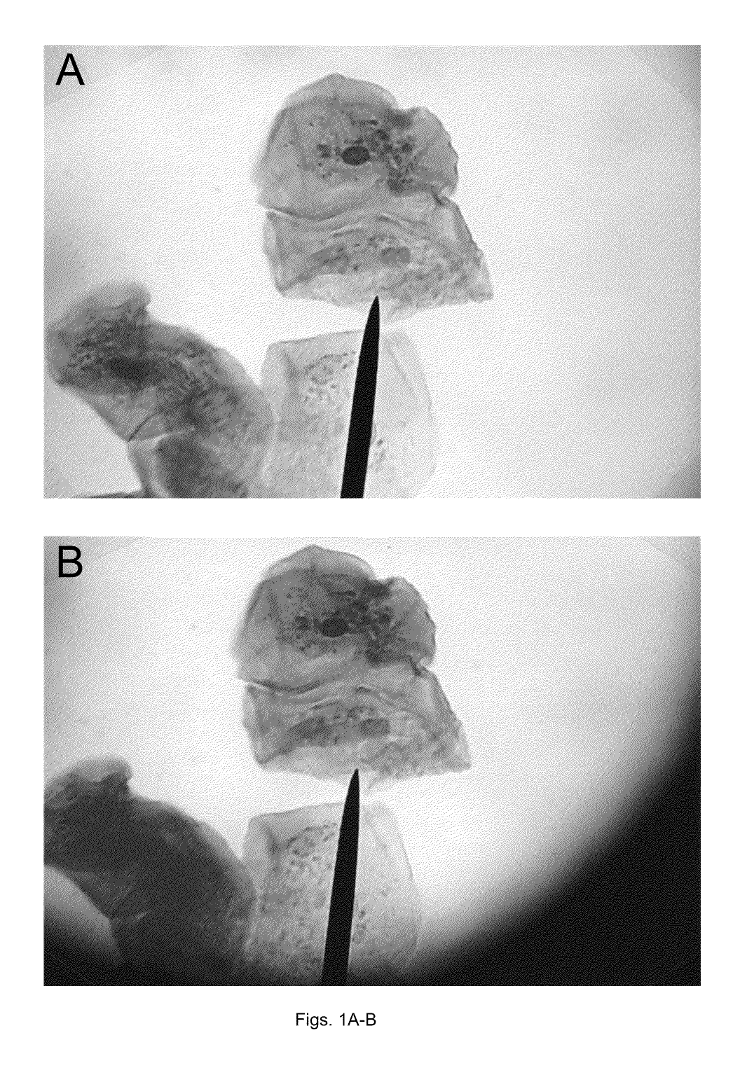

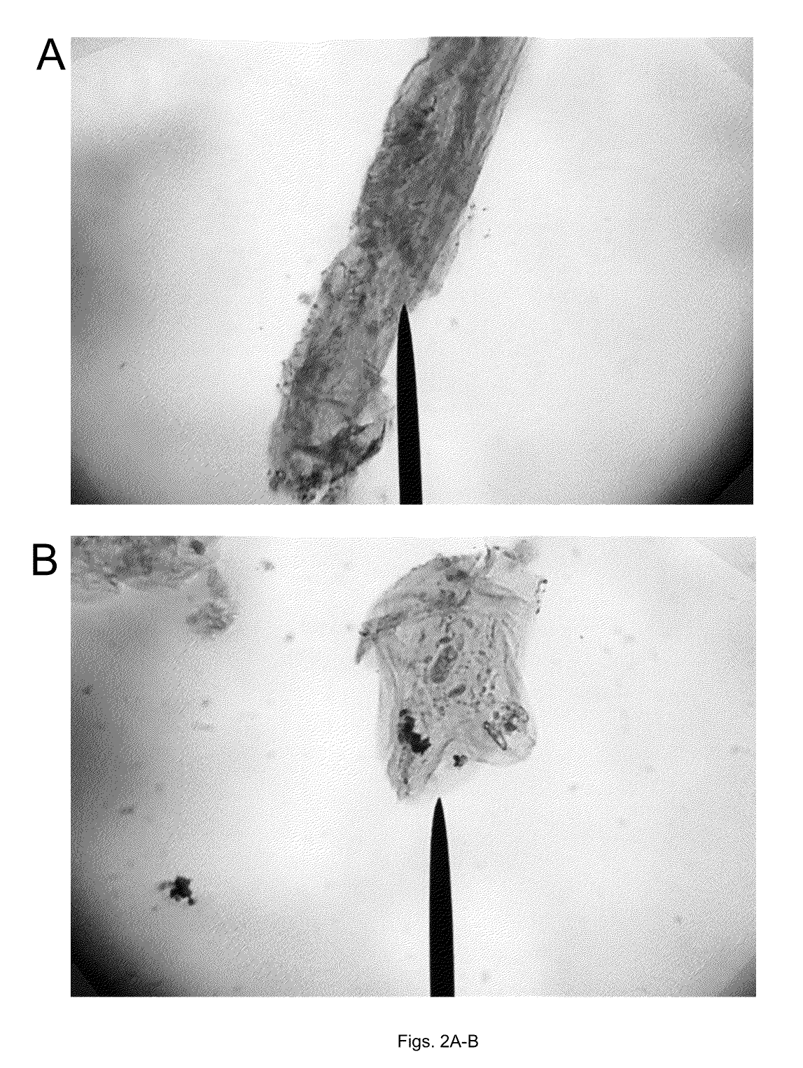

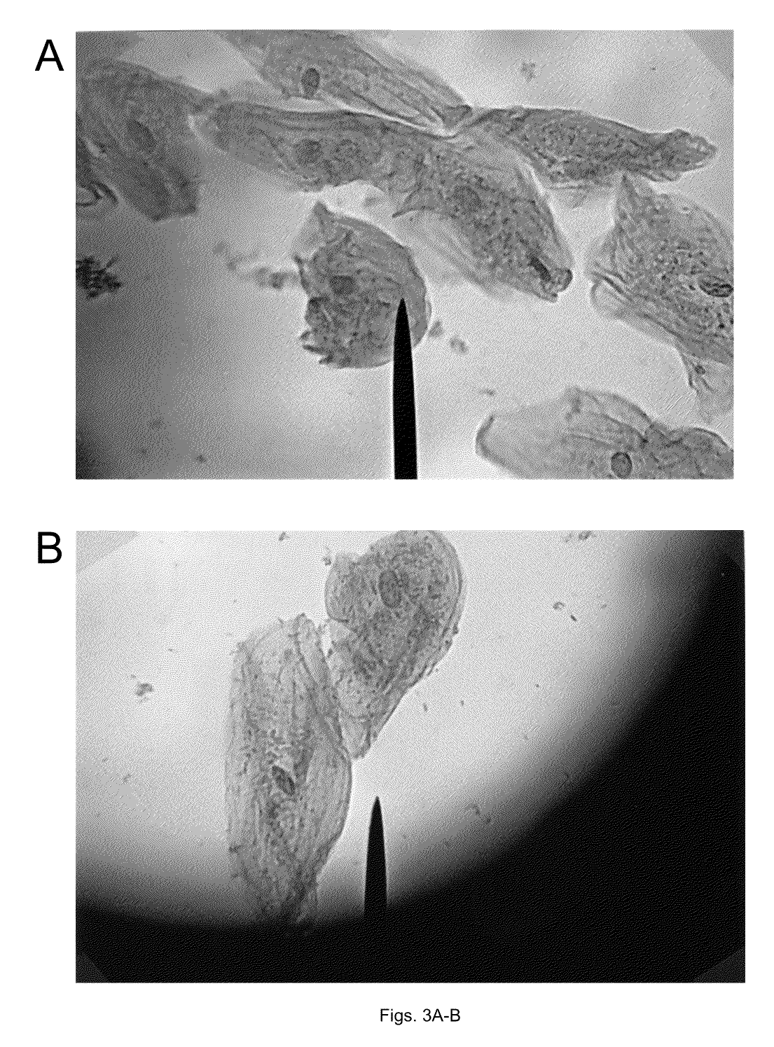

(A). Test Results of the Patent Method with Charts and Tables of the Results. As can be seen in FIGS. 1a, 1b, 2, and 3, the patent method for cleaning, concentrating and growing human cells in urine. The following is the actual protocol used.

### Methods

### Experimental Protocol

1.) A midstream urine sample of 15-30 ml is collected. Thymol as a preservative or a 24 hour collection can be taken.

2.) The sample is then refrigerated for 1-2 hours.

3.) Then immediately 10-15 of urine is placed in the 3,000-5,000 molecular weight exclusion dialysis tubing with cassette.

4.) This is then floated in 250-500 milliliters of RPMI 1640 cell culture media with glutamine and pen-strep at 100 units per milliliter.

5.) Dialyze for 24 hours.

6.) Then take the dialyzed urine (yellow color gone) and place in incubator at 37° c. with humidity.

7.) Then using a pipette taken 2-3 drops of the dialyzed urine and stain slide using giemsa stain.

8.) Begin microscopic examination using bright field or inverted microscopy to examine cells.

9.) Count the various cell types and photograph.

### Results

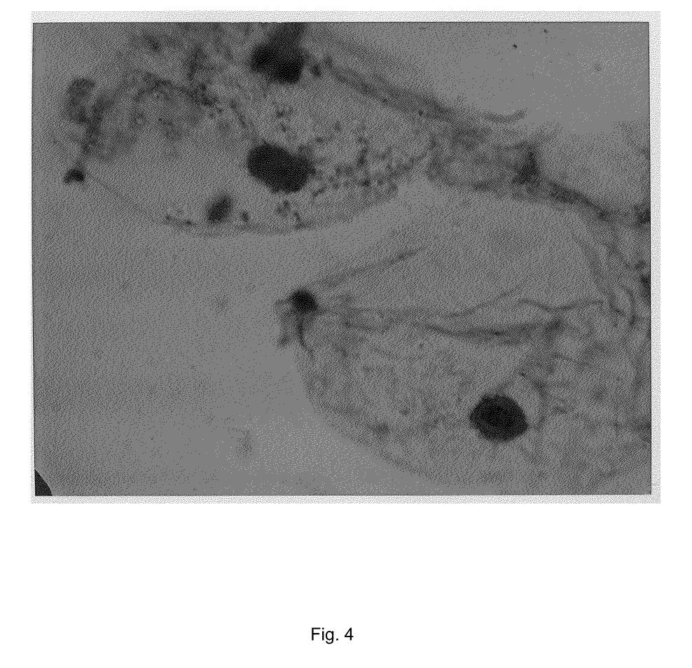

As can be seen in the photomicrographs of FIGS. 1-3, clear and well established cell types are A) clearly identifiable and B) clearly concentrated. An actual diagnostic human urine sample from a subject which had metastatic breast cancer is presented in FIG. 4.

Table 1 below identifies the cells.

| FIG. # | Identification/Description |
|---|---|
| 1 | Normal Epithelial Cells |
| 2 | Neoplastic Tissue and Cells |
| 3 | Normal Epithelial Cells with Several Neoplastic Cells |
| 4 | Metastic breast cancer/Diagnosis was determined from the |
| | following description upon cell examination: |
| | a. A cluster of neoplastic cells |
| | b. Enlarged morphology from pear shape. |
| | c. The nuclei exhibit fibrous chromatin, enlarged separated |
| | nucleoli, and thickened cell wall. |
| | d. Fibrous wavy blobs can be seen in outer edge of some cells. |
| | e. Symmetry of organelles in cytoplasm is gone and we see |
| | enlarged lacework pattern in the cytoplasm. |
| | f. the cell wall and CM is swollen and separated. |
| | g. One neoplastic cell is binucleated. |
| | h. ER around the nuclei seems to be granular. |
| | i. Vacuoles can be seen in cytoplasm. |

### Example 2

### Introduction

A comparative study is needed to compare the new and novel filtering and concentration method for urine cellular analysis (i.e., cancer cells) versus the traditional centrifugation of urine sediment seeking cells in the urine (i.e., cancer cells).

### Proposed Comparative Study

The elements of this method can be summarized in the following bulleted form.

A minimal sample group of 5 patient urines which contain suspected cancer cells will be needed.

Five (5) samples to be done by the traditional centrifugation method for examining sediment in urine, and five (5) samples (identical: one from each of the test group patients) to be done by dialysis filtration and concentration method of LifeHealth Science, LLC.

Five (5) histological slides displaying test results must be made from the traditional method and 5 histological slides made from the new and novel method.

Microscopic analysis (bright field microscopy), using giemsa stain or safranin stain should be used to analyze each of these slides at x400 to ×1000 under oil immersion, if necessary.

Each slide is to have pertinent digital photomicrographics taken for comparative analysis.

Cell counts should be taken for each type of relevant cell observed (i.e., cancer cells, squamous epithelial cells, columnar, cuboipal, bladder, kidney, prostate cells, etc.).

Each sample should be considered for cell culture to determine which method provides the cleanest, most rapid, most accurate and most easily separated cells.

Once this is considered, the question must be asked—which method culture will provide the best cancer cells to treat with experimental chemotherapies for assessment of best chemo treatment for specific patients? This comparative study should confirm that the new and novel UCT Test of LifeHealth Science, LLC provides a new and novel methodology for enhanced clarification of urine, easier identification and counting of cells, and a unique method for capturing and reproducing live cancer cells for use in research, testing and patient treatment.

While this application has been particularly shown and described with references to preferred embodiments thereof, it will be understood by those skilled in the art that various changes in form and details may be made therein without departing from the scope of the application encompassed by the appended claims. All patents, publications and references cited in the foregoing specification are herein incorporated by reference in their entirety.

Having described the invention, the following is claimed:

1. A method of isolating a cell from a urine sample comprising:
dialyzing the urine sample with a dialysate separated from the urine sample by a dialysis membrane, wherein the dialysis membrane excludes the passage of the cell through the dialysis membrane.

2. The method of claim 1, wherein the dialysis membrane is a molecular weight exclusion dialysis membrane.

3. The method of claim 2, the molecular weight exclusion dialysis membrane having a molecular weight cut off of about 3000 daltons to about 5000 daltons.

4. The method of claim 1, the dialysate comprising a cell culture medium.

5. The method of claim 1, further comprising the step of maintaining the isolated cell in culture.

6. The method of claim 1, further comprising the step of staining the cell with a cytological staining agent and identifying the cell, wherein the cell is identified using microscopic examination.

7. The method of claim 6, wherein the cytological staining agent comprises a morphological stain, the morphological stain selected from the group consisting of May-Griinwald-Giemsa stain, Giemsa stain, Papanicolau stain, Hematoxylin-Eosin stain and DAPI stain.

8. The method of claim 1, the cell comprising an abnormal cell.

9. The method of claim 1, the abnormal cell selected from the group consisting of a neoplastic cell, a tumor cell, a circulating tumor cell, a pre-cancerous cell, a cancerous cell, and a cancer cell.

10. The method of claim 9, the cancer cell selected from the group consisting of a kidney cancer cell, a bladder cancer cell, and an urethal cancer cell.

11. A method of isolating a cell from a urine sample comprising:
dialyzing the urine sample with a dialysate separated from the urine sample by a dialysis membrane, wherein the dialysis membrane excludes the passage of the cell through the dialysis membrane and has a molecular weight cut off of about 3000 daltons to about 5000 daltons.

12. The method of claim 11, the dialysate comprising a cell culture medium.

13. The method of claim 11, further comprising the step of staining the cell with a cytological staining agent and identifying the cell, wherein the cell is identified using microscopic examination.

14. The method of claim 11, the cell comprising an abnormal cell.

15. The method of claim 14, the abnormal cell selected from the group consisting of a neoplastic cell, a tumor cell, a circulating tumor cell, a pre-cancerous cell, a cancerous cell, and a cancer cell.

16. The method of claim 15, the cancer cell selected from the group consisting of a kidney cancer cell, a bladder cancer cell, and an urethal cancer cell.

17. A diagnostic assay for detecting a disorder characterized by abnormal growth in a subject comprising:
obtaining a urine sample from the subject;
dialyzing the urine sample with a dialysate separated from the urine sample by a dialysis membrane, wherein the dialysis membrane excludes the passage of a cell through the dialysis membrane;
examining the dialyzed sample, and
correlating the presence of an abnormal cell in the dialyzed sample with a disorder characterized by abnormal cell growth.

18. The method of claim 17, the step of examining the dialyzed sample comprising staining the dialyzed sample with a cytological staining agent in order to detect the presence of an abnormal cell, wherein the cell is identified using microscopic examination.

19. The method of claim 17, the dialysis membrane being a molecular weight exclusion dialysis membrane having a molecular weight cut off of about 3000 daltons to about 5000 daltons.

20. The method of claim 19, the dialysate comprising a cell culture medium.

21. The method of claim 20, the abnormal cell selected from the group consisting of a neoplastic cell, a tumor cell, a circulating tumor cell, a pre-cancerous cell, a cancerous cell, and a cancer cell.

22. The method of claim 17, the abnormal cell comprising a cancer cell, wherein the presence of a cancer cell in the dialyzed sample is indicative of a positive diagnosis of cancer.
